(19) United States
(12) Patent Application Publication
(10) Pub. No.: US 2025/0083113 A1
(43) Pub. Date: Mar. 13, 2025

(54) Apparatus for Improving Hydration and/or Reducing Particle Size of a Product and a Method of Use Thereof

(71) Applicant: St Andrews Pharmaceutical Technology Limited, Henley-on-Thames, Oxfordshire (GB)

(72) Inventor: William John Henry, St Andrews (GB)

(21) Appl. No.: 18/537,298

(22) PCT Filed: Jun. 13, 2022
(86) PCT No.: PCT/GB2022/051478

§ 371 Date: Dec. 12, 2023

(30) Foreign Application Priority Data
Jun. 14, 2021 (GB) 2108437.1
Mar. 3, 2022 (GB) 2202960.7

(51) Int. Cl. B01F 33/05; A61K 9/19; A61K 31/69; A61K 31/711; A61K 38/21; A61K 38/37; A61K 38/48; A61K 41/00; B01F 101/22; B01F 101/44; C07K 16/24; C07K 16/28; C07K 16/32; C12N 13/00

(52) U.S. Cl. CPC B01F 33/053; A61K 9/19; A61K 31/69; A61K 31/711; A61K 38/215; A61K 38/37; A61K 38/4893; A61K 41/00; C07K 16/241; C07K 16/2818; C07K 16/32; C12N 13/00; B01F 2101/22; B01F 2101/44

(57) ABSTRACT

A method and apparatus for improving hydration and/or reducing the particle size of a product or agent. The method includes the step of applying a pulsed electromagnetic field to the product or agent for a period of time sufficient to allow an increase in the hydration of the product or agent and/or a reduction of the particle size of the product or agent.

Apparatus for improving hydration and/or reducing particle size of a product and a method of use thereof.

The invention to which this application relates is to apparatus for improving the hydration of a product and/or reducing the particle size or sizes of a product and a method of using the same.

Although the following description refers almost exclusively to specific examples of the apparatus of the present invention being used for improving the reconstitution of a product which has previously been freeze-dried, and particularly to blood protein Factor VIII, it will be appreciated by persons skilled in the art that the apparatus could be used for reconstituting any freeze dried product, hydrating and/or reducing the particle size or sizes in any product and/or the like.

Freeze-drying products is a well-known process to allow products to be stored for a significantly longer period of time than would otherwise be the case, while allowing the properties of the products to be retained. The freeze-drying of proteins is also well-known and has become an important process for the preservation of biological products. There are various protocols for freeze-drying proteins which are typically followed to ensure that the freeze-dried protein product can then be stored and transported to a location for subsequent use and then reconstituted into a form to allow such use.

Freeze-drying is also known as lyophilization and acts to remove water from the frozen product by sublimation and desorption. While there are known issues with the use of freeze-drying, generally, it is regarded as a useful and preferred manner of prolonging the storage life of protein products. The process relies on the control of pressure and temperature in a lyophilizer to remove liquid from formulations that consist of thermally sensitive or hydrolytically unstable active pharmaceutical ingredients (APIs) or formulation components. The resulting solid or powder obtains greater stability than the pre-lyophilized aqueous solution, allowing the same to be stored for longer durations at higher temperatures than the pre-lyophilized aqueous solution.

During the freeze-drying process, the moisture in the form of water, which is present in the protein, is removed to a substantial extent and, in order to avoid the proteins from collapsing, the water is typically replaced with a cryoprotectant, such as for example one or more types of sugar, which in many instances provide an outer layer on the proteins.

It is found that when the reconstitution of the freeze-dried product is required, the necessity of removing the sugars and replacing the same with water, in order to reconstitute the protein, does not occur to the extent desired. This therefore means that while the proteins may be partially reconstituted, they do not return to their format prior to the freeze-drying process. This therefore can mean that the potency of the reconstituted protein is much reduced in comparison to the original protein product and, furthermore, the proteins can act in a different manner to that in which it would be expected prior to the freeze-drying process.

In particular, one problem is that the retained presence of sugars in the reconstituted product, means that the particles, can effectively stick together due to the presence of the remaining sugar and this causes aggregation of particles into larger forms. For certain products, such as for example blood protein Factor VIII, when this product is reconstituted, the aggregation of particles (typically in the form of pentamers) means that when the same is injected into the body, the body's immune system reacts adversely to the aggregated particles as being a threat to the body, developing neutralizing antibodies to the same, and therefore the body's immune system attempts to destroy or reject the Factor VIII. This problem is so severe that at present, it is believed that up to 30% of haemophiliacs who require the introduction of Factor VIII into their body on regular intervals, do in fact build-up an immunity to Factor VIII which is been reconstituted and therefore reject the same [1]. Hence the life-saving treatment benefits of the introduction of Factor VIII in patients are not obtained [2]. Notwithstanding the adverse medical effect on the patient, Factor VIII is an expensive product and therefore there is a significant expense incurred in what ultimately may be unused materials.

The conventional process for reconstituting the protein material such as Factor VIII, comprises providing a vial containing the freeze-dried Factor VIII which is effectively in the form of a powder to an end-user. The end-user is instructed to add a quantity of water into the vial and then the vial is manipulated so as to swirl the water and powder to mix the same together in the vial, with the aim being to cause the water to remove the sugar and replace the same so as to reconstitute the Factor VIII particles. It is found that this conventional process is generally insufficient to allow complete reconstitution of the Factor VIII and therefore the potency of the reconstituted Factor VIII is significantly reduced. This in turn means that the end-user may have to use and inject the Factor VIII more frequently than would otherwise be required to obtain the required beneficial medical effect.

Although the above example only relates to reconstitution of freeze dried Factor VIII, it is estimated that over 60% of biologics on the market today would not be possible without lyophilization. Furthermore, market demand for lyophilization technology will only increase as more biosimilars and novel biologics are developed. There is therefore a need in the marketplace to ensure efficient and effective reconstitution of lyophilized products.

A further problem associated with the intravenous (IV) delivery of therapeutic products is the development of Complement Activation Related Pseudo-allergy (CARPA). This is an adverse non-immune anaphylactic reaction or hypersensitivity reaction, characterised by the independence of antigen specific immune responses, against certain nanoproteins found in the IV infusion that are exposed to a patient's blood. Although CARPA for most patients is largely harmless, the reaction can be fatal in some patients and is therefore an area of concern in IV delivery of therapeutic products. One example of an agent which has been associated with the development of CARPA is Tween 80 or polysorbate 80. This agent is often used as a surfactant dispersing agent in IV formulations. Exposure of IV delivered Tween 80 in some patients results in the development of excipient aggregates (micelles or vesicles) that are thought to cause CARPA.

A yet further problem associated with large particle sizes is in transfection processes. Transfection is a technique by which extracellular matter, such as nucleic acid, can be delivered into one or more eukaryotic cells. One example of transfection is the delivery of nucleic acid using DNA plasmids. In some cases, the DNA plasmids are too large for use in a single transfection. As such, a number of transfection steps have to be used using smaller DNA plasmids. This results in more time consuming processes that are much expensive to carry out. In addition, the overall transfection frequency is low.

It is therefore an aim of the present invention to provide apparatus to improve the reconstitution process of a freeze-dried or lyophilized product in such a way so as to allow the reduction or elimination of aggregation of the product particles during the reconstitution process and/or to increase the potency of the reconstituted product.

It is a further aim of the present invention to provide a method of improving the reconstitution process of a freeze dried or lyophilized product.

It is a yet further aim of the present invention to provide apparatus and/or a method to improve hydration of a product in such a way so as to allow the reduction or elimination of aggregation of product particles.

It is a yet further aim of the present invention to provide apparatus and/or a method to mitigate CARPA reactions in IV delivered products or formulations.

It is a yet further aim of the present invention to provide apparatus and/or a method to reduce the size of DNA plasmids to allow improvement of transfection frequency.

According to a first aspect of the present invention, there is provided a method of improving hydration and/or reducing the particle size of a product or agent, said method including the step of applying a pulsed electromagnetic field to the product or agent for a period of time sufficient to allow an increase in the hydration of the product or agent and/or a reduction of the particle size of the product or agent.

The method of the present invention has the advantage that it reduces the particle size of the product or agent and/or mitigates or reduces the formation of aggregates in the product or agent. This will increase the potency of the product or agent and improve functionality and/or performance of the same.

It will be appreciated that reference to applying a pulsed electromagnetic field also covers exposure to and/or the like.

In one embodiment the period of time to which the product or agent is exposed to the pulsed electromagnetic field is a pre-determined period of time. Further preferably this pre-determined time is 10-15 minutes+/−5 minutes.

In one embodiment the product or agent is media or cell media.

In one embodiment the product or agent is a lyophilized or freeze-dried product or agent. Preferably the method is carried out during reconstitution of the lyophilized product or agent.

In one embodiment the product or agent is a powdered or granular product or agent.

Preferably the method of reconstitution of the lyophilized or freeze-dried product or agent includes the step of adding water or liquid to the lyophilized or freeze-dried product or agent, applying the pulsed electromagnetic field to the mixture of water or liquid and lyophilized or freeze-dried product or agent for a period of time sufficient to allow an increase in the hydration of the product or agent and/or a reduction of the particle size of the product or agent.

In one embodiment, in addition to applying the pulsed electromagnetic field to the mixture, product or agent, the mixture, product or agent may be agitated to cause mixing of the lyophilized or freeze-dried product or agent and water or liquid, or agitation of the product, liquid or mixture, at any time prior to the exposure to the pulsed electromagnetic field, during the exposure to the pulsed electromagnetic field and/or after exposure to the pulsed electromagnetic field.

Preferably the step of agitation is undertaken using agitation means or an agitation device, such as for example, a magnetic stirrer, a vibration mechanism and/or the like.

In one embodiment, the pulses of the pulsed electromagnetic field are generated in a predetermined manner which may comprise or consist of a sequence of pulses all of the same duration and frequency and/or may comprise or consist of a sequence of pulses of different durations and/or at different frequencies.

In one embodiment, the said mixture is exposed to the pulsed electromagnetic field for the duration of the reconstitution of the said product or agent in the mixture.

In one embodiment, the addition of water or liquid to the said product or agent to form the mixture occurs whilst the water, liquid and/or mixture is exposed to the pulsed electromagnetic field.

In one embodiment, the water or liquid used for reconstitution is exposed to the pulsed electromagnetic field prior to addition with the lyophilized or freeze-dried product or agent.

In one embodiment, the product, agent or mixture is located within a container which can be integral with a device from which said pulsed electromagnetic field is generated, or which is separate to and placed on, in or adjacent a device from which said pulsed electromagnetic field is generated.

In one embodiment, the said container is that in which the freeze-dried or lyophilized product or agent is initially provided and into which the water or liquid is added.

Typically, the pulsed electromagnetic field which is generated is sufficient to cause rotation of water particles and the effect of the rotation is to aid the removal of one or more sugars which may be present on the freeze-dried or lyophilized product or agent. This improves the level of removal of the sugars from the product and replacement by the water particles to thereby improve the potency of the reconstituted product or agent and reduce the tendency of aggregation of the product or agent during the reconstitution process.

In one embodiment, when the potency of the reconstituted product or agent is increased in accordance with the invention, the frequency of usage of the reconstituted product or agent can be reduced and/or the quantity of the reconstituted product or agent at each use can be reduced.

In one embodiment, when aggregation of the reconstituted product or agent is reduced in comparison to the conventional processes, then the tendency of rejection of the reconstituted product or agent is reduced.

In one embodiment, the product or agent is a protein product, such as for example a monoclonal AB, a hormone, a fusion protein, protein constructs and/or the like.

In one embodiment the product or agent is any or any combination of trastuzumab; pembrolizumab; Infliximab; Daxibotulinumtoxin; Immunoglobulin; Omalizumab; Abatacept; Secukinumab; Interferon beta 1a, Bortezomib and/or the like.

In one embodiment, the product, agent, lyophilized or freeze-dried product or agent is DNA or a DNA plasmid.

In one embodiment, the product, agent, lyophilized or freeze-dried product or agent is Factor VIII.

In one embodiment the product or agent is or forms part of an Intravenous (IV) formulation, product or agent. Preferably the product or agent is one that is conventionally known to be associated with CARPA reactions.

In one example the product or agent is a dispersing agent, such as Tween 80 or polysorbate 80.

In one embodiment the product, mixture or agent is exposed to the pulsed electromagnetic field during an infusion or injection process into a patient. Preferably the part of the process which is exposed to the pulsed electromagnetic field can be an in vitro part and/or an in vivo part.

In one embodiment the product or agent is exposed to the pulsed electromagnetic field during or before location in an IV bag or IV delivery vessel.

Preferably the step of applying the pulsed electromagnetic field or signals takes place at room temperature (such as for example 20° C.) or takes place in an incubator that can be set at temperatures above room temperature (such as for example at 37° C.).

Preferably the pulsed electromagnetic field or signals are generated by one or more electronic devices, apparatus and/or circuits.

Preferably the one or more electronic devices, apparatus and/or circuits include transmission means or device for generating and/or transmitting the pulsed electromagnetic field or signals therefrom in use.

Preferably the transmission means or device includes one or more electronic transmission chips, the one or more electronic transmission chips arranged to generate, emit and/or transmit one or more pulsed electromagnetic signals in use.

In one embodiment reference to the transmission means or one or more electronic transmission chips could include one or more transmitters, at least one transmitter and at least one receiver, or one or more transceivers. Thus, in one example, the pulsed electromagnetic field or signals could be transmitted from a central location or a master transmitter and could be received by one or more remote and/or slave receivers and/or transceivers for subsequent re-transmission or emission therefrom.

In one embodiment the electronic device has a single transmission means or electronic transmission chip. Such a single transmission means or electronic transmission chip is sufficient to provide a pulsed electromagnetic field or signal to the product or agent. In one exemplary embodiment, a single transmission means or electronic transmission chip is provided attached or integrated into a container for containing the product or agent in use.

In one embodiment the electronic device has two or more transmission means or electronic transmission chips. Preferably the two or more transmission means or electronic transmission chips are arranged a pre-determined spaced distance apart from each other, such as for example in the electronic device.

Preferably the pre-determined spaced distance apart is such so as to provide the product or agent being pulsed with the electromagnetic pulsed field or signals a desired effect (i.e. to reduce the particle size and/or improve hydration) and/or to provide an even or substantially even distribution of electromagnetic radiation/signals in use.

Preferably the electronic device has a plurality of transmission means or electronic transmission chips arranged in a pre-determined pattern and/or array.

Whilst a single transmission means or electronic transmission chip is sufficient to provide the advantageous properties of the invention, it has been found that having a plurality of transmission means or electronic transmission chips allows the pulsed electromagnetic field or signal to be delivered across a broader range of surface areas whilst still maintaining a maximal effect. Applicants have found that having a transmission means or electronic transmission chip evenly distributed such that there is at least one chip per 18.5 cm² provides sufficient coverage for the optimal effect.

In some embodiments, the electronic device, circuit or apparatus comprises one or more transmission means or electronic transmission chips. In some embodiments, the apparatus comprises 2, 3, 4, 5, 6, 7, 8, 9, or 10 or more transmission means or electronic transmission chips.

In some embodiments, there is one transmission means or electronic transmission chip per approximately 105 to 115 cm² of a surface of the housing of the apparatus, device or a surface of an item as defined herein, and preferably approximately 110 cm² of a surface of the housing of the apparatus, device or a surface of an item as defined herein.

In some embodiments, there is one transmission means or electronic transmission chip per approximately 50 to 60 cm² of a surface of the housing of the apparatus, device or a surface of an item as defined herein, and preferably approximately 55 cm² of a surface of the housing of the apparatus, device or a surface of an item as defined herein.

In some embodiments there is one transmission means or electronic transmission chip per approximately 25 to 30 cm² of a surface of the housing of the apparatus, device or a surface of an item as defined herein, and preferably approximately 27.5 cm² of a surface of the housing of the apparatus, device or a surface of an item as defined herein.

In some embodiments there is one transmission means or electronic transmission chip per approximately 15 to 20 cm² of a surface of the housing of the apparatus, device or a surface of an item as defined herein, and preferably approximately 18.5 cm² of a surface of the housing of the apparatus, device or a surface of an item as defined herein.

In some embodiments, there is one transmission means or electronic transmission chip per approximately 10 to 15 cm² of a surface of the housing of the apparatus, device or a surface of an item as defined herein, and preferably approximately 12.2 cm² of a surface of the housing of the apparatus, device or a surface of an item as defined herein.

In an exemplary embodiment, six transmission means or electronic transmission chips are provided in the apparatus, device and/or circuit.

In one embodiment, where more than one transmission means or electronic transmission chip is required, the spacing of the plurality of transmission means or electronic transmission chips must be optimised. In order to achieve an optimal pre-determined space between each transmission means or electronic transmission chips, the transmission means or electronic transmission chips should be positioned at a distance equal or substantially equal to half the wavelength of the electromagnetic radiation frequency being used. Preferably this distance should be considered to be relevant in any plane of orientation or two or more transmission means or electronic transmission chips being used together as part of the apparatus or device. For example, if the wavelength is 12.4 cm, the transmission chips should be placed approximately 6.2 cm apart to produce an optimal electromagnetic field when in use.

In one example, the pre-determined spaced distance=wavelength/2.

In one example, the pre-determined spaced distance in the X-axis and/or Y-axis is half the wavelength between each transmission means or electronic transmission chip in an evenly spaced grid. Such an arrangement minimises the risk of destructive interference.

In one embodiment the electronic device includes a housing and the one or more transmission means or transmission chips are located in said housing.

Preferably the housing includes at least one flat or planar surfaces to allow the housing to be located in a stable manner with respect to the one more items or container receiving the pulsed electromagnetic field or signals in use. Alternatively, the housing can include one or more curved or non-planar surfaces to allow the housing to be located in a stable manner with respect to one or more items or containers receiving the pulsed electromagnetic field or signals in use.

In one example, at least one surface of the housing includes one or more recesses for the location of the one or more items receiving the pulsed electromagnetic field or signals in use.

In one embodiment the housing includes a base surface for allowing the housing to be supported directly or indirectly on a surface in use. Further preferably the housing includes an upper surface opposite to the base surface. Preferably the upper surface is the surface on which the one or more items receiving the pulsed electromagnetic field or signals can be positioned in use.

In one embodiment the electronic device and/or housing is attachable to an external surface of a container, reactor vessel and/or the like. For example, the electronic device and/or housing can be attachable via one or more attachment means or device including any or any combination of one or more screws, nuts and bolts, magnets, ties, clips, straps, inter-engaging members, adhesive, welding and/or the like.

Preferably the upper surface of the housing and/or the distance between the transmission means and the one or more items, containers, products or agents receiving the pulsed electromagnetic signals when located on, in or relative to the housing or electronic device in use is approximately 25 cm or less, 20 cm or less, 15 cm or less, 10 cm or less or 5 cm or less. Further preferably the distance is approximately 1 cm.

Preferably the pulsed electromagnetic field or signals are provided in a pre-determined sequence of pulses.

In one embodiment the pulsed electromagnetic signals or field is provided at a frequency in the range of approximately 2.2-2.6 GHz and, further preferably the pulsed electromagnetic signals are transmitted at a frequency of approximately 2.4 GHz+/−50 MHz or more preferably 2.45 GHz+/−50 MHz.

In one embodiment the pulsed electromagnetic signals or field is provided at a frequency within the range of the industrial, scientific and medical radio frequency band (ISM band) of 2.4 to 2.4835 GHz, preferably 2.45 GHz+/−50 MHz.

Preferably the pulsed electromagnetic signals or field are pulsed at a frequency of approximately 50 Hz or less, further preferably approximately 25 Hz or less, and yet further preferably approximately 15 Hz or less.

Preferably each pulse of the pulsed electromagnetic signals or field lasts for between approximately 1 ms-20 ms. Further preferably each pulse lasts for approximately 1 ms.

Preferably the time period between pulses (also referred to as the “rest period” or “relaxation period”) is approximately 66 ms or less.

Preferably the duty cycle of the pulsed electromagnetic signals is less than 2%.

In one embodiment the transmission power provided by each transmission means or chip in the electronic device is 2 dBm-+4 dBm, approximately 1 mW, approximately 2 mW or approximately 2.5119 mW.

In one embodiment the pre-determined frequency of the pulsed electromagnetic field or signals is approximately 2.2-2.6 GHz, 2.4 GHz+/−50 MHz or 2.45 GHz+/−50 MHz, the pre-determined pulse rate is approximately 15 Hz or has a duty cycle of less than 2%, and the pre-determined power is +2 dBm-+4 dBm, 1 mW, 2 mW or 2.5119 mW.

Preferably the pulsed electromagnetic field or signals are transmitted using Gaussian Frequency Shift Keying (GFSK) between 0.45 and 0.55.

Preferably the pulsed electromagnetic field or signals are radio frequency (RF) data signals.

Preferably the pulsed electromagnetic field or signals is a digital sequence of pulsed electromagnetic signals.

Preferably the radio frequency field or signals utilize the Bluetooth LE (BLE) protocol's advertising feature. Preferably the advertising RF signals are on channels 37, 38 and 39 corresponding to frequencies 2402 MHz, 2426 MHz, 2480 MHz respectively.

Preferably the pulsed electromagnetic signals are directed towards one or more DNA plasmids for transfection, a lyophilized product or agent for reconstitution or being reconstituted, an IV formulation, product or agent and/or the like.

In one embodiment the electronic device includes power supply means for supplying electrical power to the device in use. Preferably the power supply means includes a mains electrical power supply, one or more batteries, power cells, one or more rechargeable batteries, electrical generator means and/or the like.

In one embodiment the electronic device includes control means for controlling operation of the electronic device and/or transmission means in use.

In one embodiment the electronic device includes one or more circuit boards. Preferably the transmission means can be provided on the one or more circuit boards, typically in the form of an integrated circuit, and/or other components, such as for example memory means, are located.

In one embodiment the electronic device includes memory means, such as a memory device, data storage device and/or the like.

Preferably the other components of the electronic device includes one or more components required for the selective operation of the apparatus and, when active, the controlled operation of the same to generate the pulsed electromagnetic signals. For example, user selection means can be provided on the device to allow user selection of one or more conditions, operation and/or one or more parameters of the device in use; display means to display one or more settings, options for selection and/or the like.

In one embodiment the said further components or power supply means include one or more power cells and the same may all be contained within the housing.

In one embodiment the housing of the electronic device is provided in a form which allows the same to be engaged with and/or located with respect to a container in which the material and/or one or more items which is to be exposed to the electromagnetic signals is located in use.

In one embodiment the control means includes an option to allow the user to select any or any combination of the signal frequency, signal strength, signal power, signal pulse rate, time period of signal pulsing, and/or the like of the said pulsed electromagnetic signals. In one embodiment the selection of the frequency, strength, power, pulse rate, time period of pulsing, other parameters and/or the like may be made with respect to the particular form of the agent or product and/or one or more containers which are to be exposed to the pulsed electromagnetic field or signals in use, the quantity of said product or agent, the dimensions of the container with respect to which the apparatus is located for use and/or other parameters.

In one embodiment, the apparatus includes agitating means or device to allow agitation of the product, mixture or agent in use.

In one embodiment, the electronic device generating and/or emitting the pulsed electromagnetic field includes means or device for measuring the turbidity and/or particle size of the product or agent which is to be exposed to the pulsed electromagnetic field. For example, the device could include light scattering means, a nephelometer means and/or the like. The light scattering means and/or turbidity measuring means could be used as an indication of the presence of aggregates, micelles, vesicles, particles and/or the like in the product, mixture or agent.

In one example, the electronic device could include a laser or columnated light source located on one side of location means where the product or agent is to be located in use, and a light detector could be located on the opposite side of the location means. The light detector detects the amount of light passing through the product, mixture or agent from the light source as a means of determining turbidity and/or particle size of the product, mixture or agent in use.

In a further example, a second light detector could be located transverse to, perpendicular or substantially perpendicular to the light source and/or first light detector. A comparison of the light detected from the first and second light detectors could be used to determine the turbidity and/or particle size of the product, mixture or agent in use.

Typically the means for measuring the turbidity and/or particle size of the product, mixture or agent which is to be exposed to the pulsed electromagnetic field can be integral with the electronic device, can be attached or detachably attached to the electronic device or can be associated with the electronic device.

In one embodiment the electronic device is arranged to generate and/or emit the pulsed electromagnetic field for a period of time sufficient to reduce the measure of turbidity and/or particle size of the product, mixture or agent below a pre-determined threshold level.

In one embodiment the electronic device could include audio, visual and/or kinaesthetic means for signalling to a user when the product, mixture or agent being exposed to a pulsed electromagnetic field has fallen below the pre-determined threshold level for turbidity and/or particle size required. Once the user is signalled, the user will know the product or agent is safe to injection, infuse and/or the like.

For example, a red light could be shown on the electronic device when the product or agent detects the turbidity and/or particle size of the product, mixture or agent is above a pre-determined threshold level and should not be used by the user, and a green light could be shown on the electronic device when, following exposure to the pulsed electromagnetic field, the device detects the turbidity and/or particle size of the product, mixture or agent is below a pre-determined threshold level and can be used by the user.

In one example, one or more audio signals, such as a “ping” or alarm could be emitted when an agent, mixture or product is detected as being safe to use following exposure to the pulsed electromagnetic field.

In one example, one or more vibrations could be emitted from the device when an agent, mixture or product is detected as being safe to use following exposure to the pulsed electromagnetic field.

According to a second aspect of the present invention, there is provided apparatus for improving hydration and/or reducing the particle size of a product or agent in use, said apparatus arranged to generate and emit a pulsed electromagnetic field capable of being directed towards a product or agent for a time sufficient to allow an increase in the hydration of the product or agent and/or a reduction of the particle size of the product or agent.

Preferably the apparatus includes means or device for detecting particle size and/or turbidity of the product or agent.

According to one aspect of the present invention there is provided a reconstituted product or agent which has been reconstituted utilising the application of a pulsed electromagnetic field to which the said product or agent has been applied for a period of time.

According to one aspect of the present invention there is provided an intravenous product or agent suitable for intravenous delivery to which a pulsed electromagnetic field has been applied for a period of time.

According to one aspect of the present invention there is provided a DNA plasmid to which a pulsed electromagnetic field has been applied for a period of time.

According to a further aspect of the invention, there is provided a method for reconstituting of a lyophilized or freeze-dried product or agent, said method including the steps of adding water to the lyophilized or freeze-dried product or agent to create a mixture, operating one or more devices to generate a pulsed electromagnetic field, placing the mixture within the pulsed electromagnetic field for a period of time so as to allow the reconstitution of the said product or agent.

According to a further aspect of the invention, there is provided a method for preparing an intravenous product or agent suitable for intravenous delivery in a patient in use, said method including the steps of applying a pulsed electromagnetic field to the intravenous product or agent for a period of time sufficient to reduce or prevent the formation of excipient aggregates.

Preferably the excipient aggregates are micelles and/or vesicles.

According to a yet further aspect of the present invention there is provided a method of reducing the size of a DNA plasmid, said method including the steps of applying a pulsed electromagnetic field to the DNA plasmid for a period of time sufficient to reduce the size of the DNA plasmid.

Specific embodiments of the invention are now described with reference to the accompanying drawings wherein:

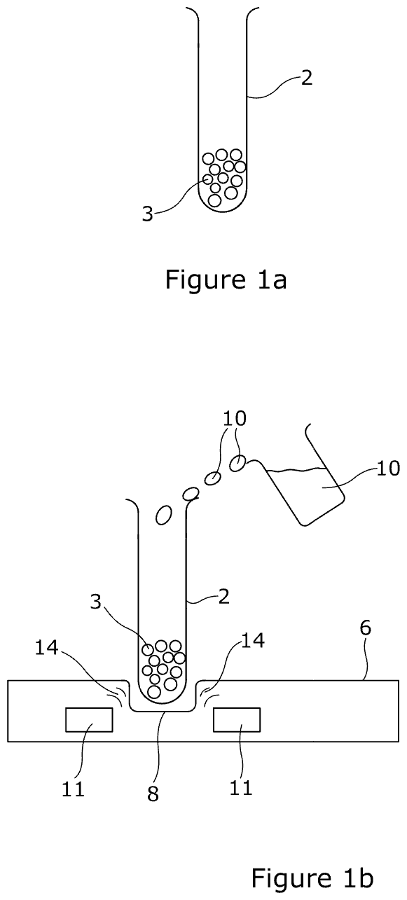

FIGS. 1a and 1b illustrate schematically, apparatus in accordance with one embodiment of the invention;

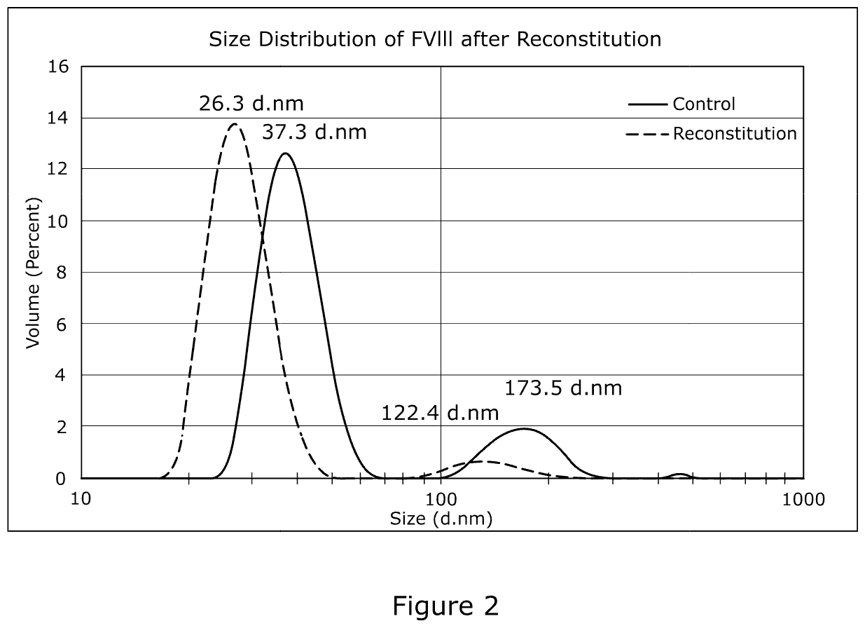

FIG. 2 illustrates results data obtained from the use of conventional reconstitution process (Control) and a reconstitution process in accordance with one embodiment of the present invention for Factor VIII;

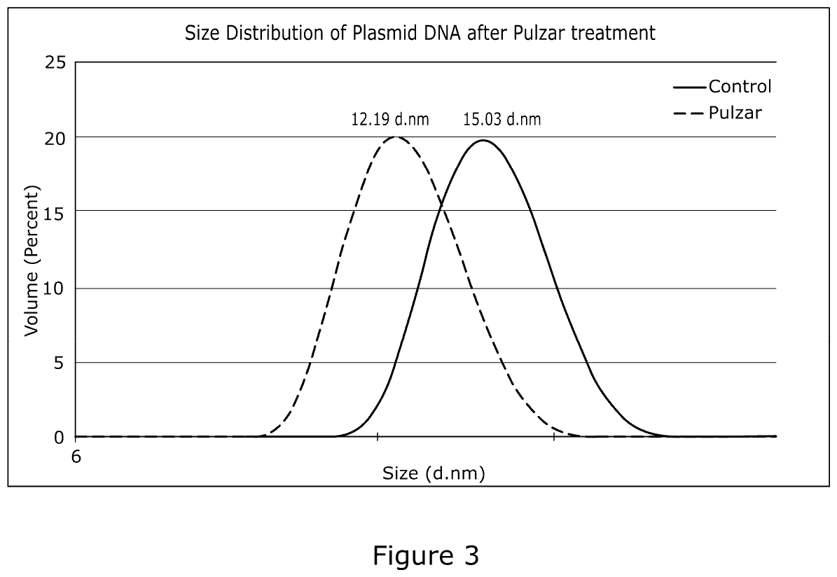

FIG. 3 illustrates result data obtained for the measurement of the size of DNA plasmids with and without (control) being exposed to a pulsed electromagnetic field according to an embodiment of the present invention;

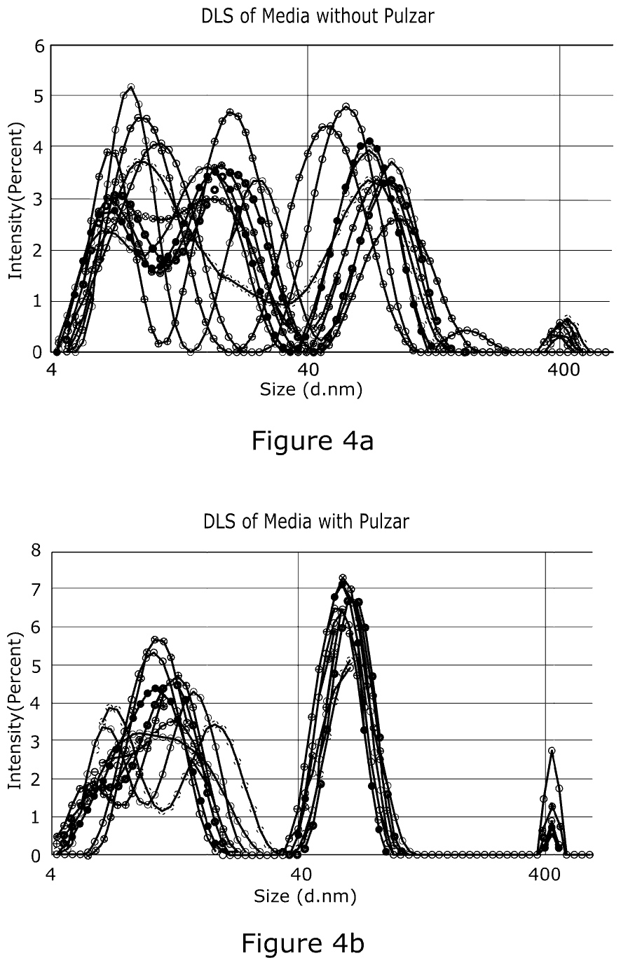

FIG. 4a and 4b illustrate results data obtained for the use of a pulsed electromagnetic field on media according to an embodiment of the present invention, wherein figure da shows the control where the media was not exposed to a pulsed electromagnetic field and FIG. 4b shows present invention where the media was exposed to a pulsed electromagnetic field;

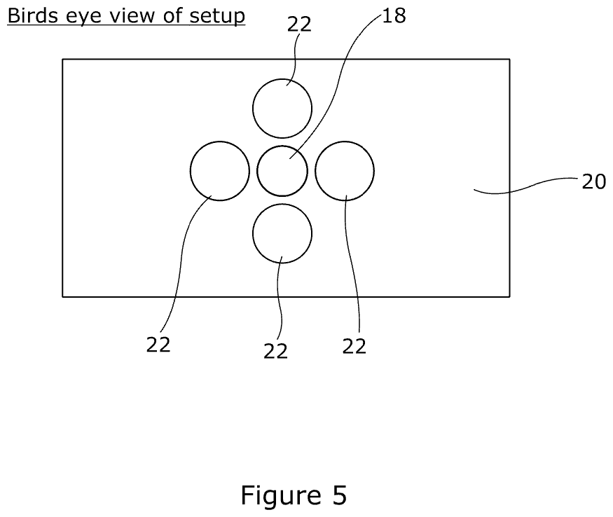

FIG. 5 is a top plan view of the Experimental Arrangement of the Pulsed electromagnetic field devices for the Active Samples in Experiment 6;

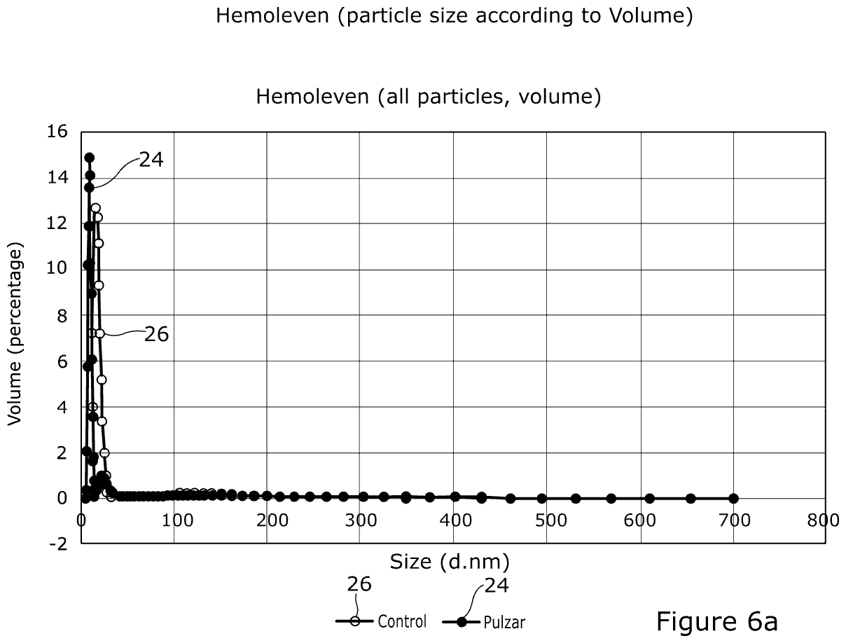

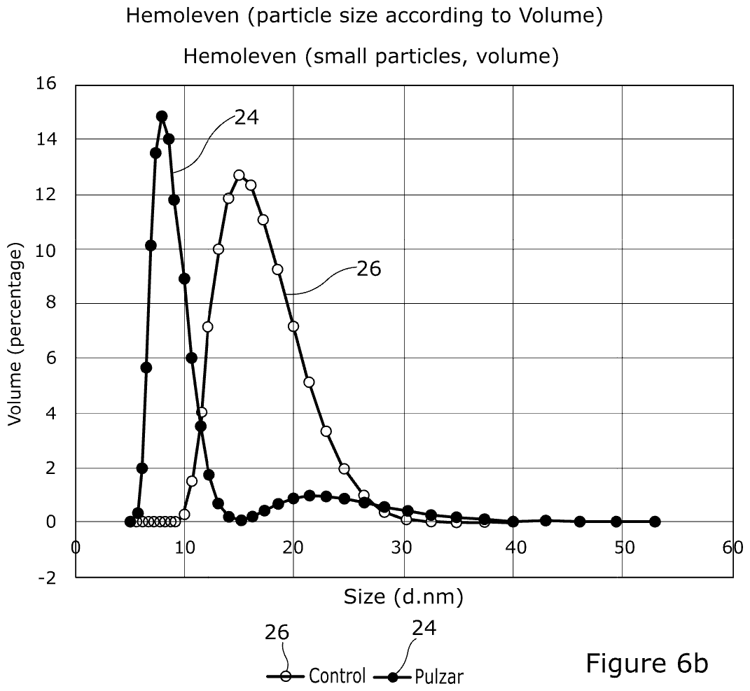

FIGS. 6a and 6b show results data obtained from the use of conventional reconstitution process (Control) and a reconstitution process in accordance with an embodiment of the present invention for Factor XI. FIG. 6a shows all the particle sizes and FIG. 6b is an enlarged view showing the smaller sized particles in FIG. 6a;

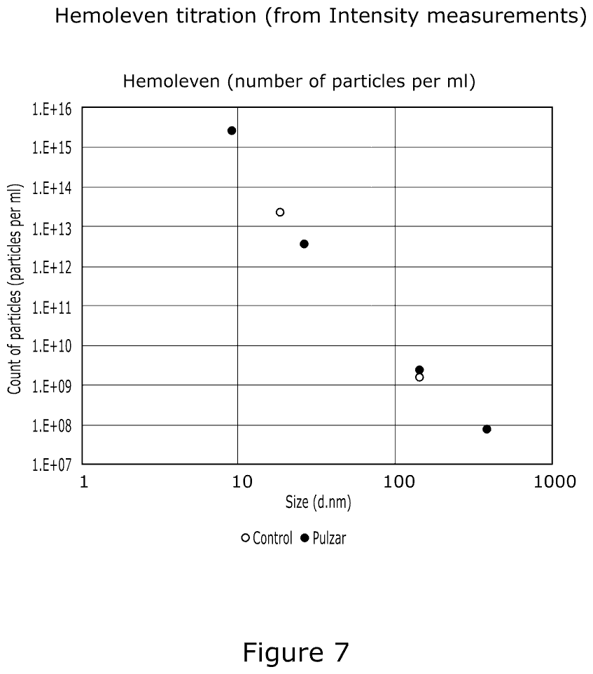

FIG. 7 illustrates results data for Factor XI in experiment 7 with the particle size against the number of particles per ml.

Referring firstly to FIG. 1, there is illustrated apparatus in accordance with one embodiment of the invention for use in the reconstitution of a freeze-dried product or agent.

The apparatus includes a container, in this case in the form of a vial 2 which has a cavity in which a freeze-dried product 3, such as Factor VIII, is provided and contained. In one embodiment, this vial may be used to transport the said Factor VIII in a freeze-dried condition from a location at which the freeze-drying occurs to an end-user location which may be at a domestic premises and at which, it will be appreciated, the end-user has limited or no apparatus available to them. Thus, in accordance with the invention, there is provided apparatus which can be used by the non-skilled personnel to allow the improved reconstitution of the freeze-dried product. In the embodiment shown, the vial 2, with the freeze-dried product 3 located therein, is opened and placed onto a plate 6 which has a locating recess 8 and into which the vial is placed. A source of water 10 is then provided which allows the water to be poured into the vial to a predefined extent and if required, some physical mixing of the contents of the vial may be performed or, alternatively, the plate may be provided with agitation means which allow the plate to effectively vibrate and in turn perform a mixing action on the water and the product within the vial. At the same time as mixing or before or after the same, a pulsed electromagnetic field generating apparatus 11 which, in this embodiment, is formed in combination with the plate 6, is operated so as to generate a pulsed electromagnetic field indicated by reference 14, which is emitted from the device, through the plate, through the container, as indicated. This therefore ensures that the water particles within the container and the product within the container, are exposed to the pulsed electromagnetic field and which causes the water particles to rotate and it is believed that the rotation action on the sugars coating of the freeze-dried protein, allows the effective removal of the sugars from the surface and, in turn, allows greater replacement of the sugars by the water particles which, in turn, allows a greater level of reconstitution of the product back to its form prior to freeze-dry. This also reduces the chance and opportunity of the particles of the product to stick together and thereby reduces the possibility of aggregation of the product.

When the product has been mixed, the same can then be removed from the container for use by the person, such as for example via an injection. The product which is injected, is closer to the condition expected by the patient's body and in which case, the level of immune rejection amongst users of the product is greatly reduced so effectively the product has achieved an immune silence condition which would not conventionally be achieved using conventional reconstitution processes.

### Experiment 1—Reconstitution of Freeze Dried Factor VIII

An experiment was undertaken to measure the effect of use of the apparatus of the present invention on particle size of reconstituted freeze dried Factor VIII.

The particle size of the blood protein product Factor VIII (FVIII) was measured using a dynamic light scattering instrument, which in this example is a Zetasizer Ultra (Malvern Panalytical Ltd, UK).

The experiment was performed on a control sample where no pulsed electromagnetic field was applied to the FVIII, and on a sample of the present invention to which a pulsed electromagnetic field was applied to the FVIII.

The method steps were as follows:
1. A vial of freeze dried Factor VIII was reconstituted with Water for Injection (WFI) as per vial instructions (swirling gently until all the visible material is dissolved and liquid looks clear).
2. 1 ml of the sample was added into a clean cuvette (part numbers PCS8501, PCS1115, or DTS0012, Malvern Panalytical, United Kingdom) for each of the control sample and the invention sample.
3. The invention sample was treated in a pulsed electromagnetic field (using PulzFector device which generates the pulsed electromagnetic field, St. Andrews Pharmaceuticals Technology Ltd, UK) by placing the cuvette into the PulzFector and turning the device on for 10-15 minutes.
4. The control cuvette and the invention cuvette were placed in turn in the Zetasizer Ultra (Malvern Panalytical, United Kingdom) and the particle size of the reconstituted FVIII was measured.

The Pulzfector device used to carry out the experiment, in one example, was an electronic device or apparatus including 6 electronic chips capable of emitting a pulsed electromagnetic field at a frequency in the range of 2.2-2.6 GHz, at a pulsed frequency of 50 Hz or less, with each pulse lasting for between 1 ms-20 ms, and the time between each pulse being approximately 66 ms or less, with a power of 2 dBm-+4 dBm.

The results of Experiment are shown in FIG. 2.

FIG. 2 is a graph showing the diameter of the particle size in nanometers (nm) on the X-axis and the percent volume on the Y Axis. The particle size distribution is represented by a solid black line for the invention sample and is represented by a dotted black line for the control sample. In the control sample, two large peaks are identified for particles having a diameter of 37.3 nm and 173.5 nm. These are typically pentameric aggregates that are known to be immunogenic. In the invention sample, peaks are identified for particles having a diameter of 26.3 nm and 122.4 nm.

Thus, it can be concluded that the particle size of the reconstituted FVIII is significantly reduced and the aggregates are dispersed when exposed to a pulsed electromagnetic field compared to when no pulsed electromagnetic field is applied. The present invention therefore mitigates and reduces the formation of immune provoking protein aggregates.

The observation of a reduction in particle size in the method of reconstitution using the pulsed electromagnetic field of the present invention is thought to occur due to increasing the uniformity of hydration of the product. This creates smaller hydration spheres, which show as smaller particles using the light scattering instrumentation.

It will be appreciated that the water (WFI) used for reconstitution of the freeze-dried product or agent could be exposed to the pulsed electromagnetic field in addition to or instead of exposing the freeze-dried product or agent. The pulsed electromagnetic field could be directed at the mixture of water and freeze-dried product or agent during or after reconstitution.

### Experiment 2—Measurement of the Potency of Reconstituted Freeze Dried Factor VIII

The potency of the reconstituted FVIII in the formation of blood clots can be analysed using a one stage chromogenic assay. The method was as follows:
1. An equal volume (most commonly 0.1 ml) of the test or reference plasma was incubated at 37° C. In addition, an equal volume of Factor VIII deficient in plasma was incubated at 37° C. NOTE: The reference plasma was used to derive the concentration/activity of the test plasma by comparing clotting times.
2. Platelet poor plasma (PPP) was incubated at 37° C. with phospholipid and a contact activator was added followed by Calcium (initiates clotting). NOTE: All reagents were pre-warmed to 37° C.
3. The clotting time was measured.

It is expected that the clotting time will be significantly reduced in the invention sample which has been exposed to a pulsed electromagnetic field compared to the control sample, based on the data results obtained in Experiment 1. Thus, it can be concluded that there is an improvement in protein functionality and/or performance as a result of application of pulsed electromagnetic field to the product or agent.

### Experiment 3—Mitigation of CARPA Reactions in IV Delivered Products

This experiment was undertaken to measure the effect of use of the apparatus of the present invention on the formation of excipient aggregates (such as micelles or vesicles) on IV delivered products or formulations.

The particle size of aggregates in the IV products or formulations was measured using a dynamic light scattering instrument, which in this example is a Zetasizer Ultra (Malvern Panalytical Ltd, UK), as per the protocol in Experiment 1.

It is expected that formation of excipient aggregates in the IV products or formulations will be significantly reduced on exposure to a pulsed electromagnetic field according to the present invention, based on the data results in Experiment 1. The potency, functionality and/or performance of the IV products or formulations is likely to be significantly improved as a result of application of pulsed electromagnetic field to the IV product or agent.

### Experiment 4—Reduction in the Size of DNA Plasmids in Transfection

This experiment was undertaken to demonstrate a reduction in the size of DNA plasmids used in transfection processes using a pulsed electromagnetic field according to the present invention.
1. A transfection process was undertaken using control DNA plasmids that were not exposed to a pulsed electromagnetic field.
2. The success of the transfection process was measured.
3. A further transfection process was undertaken using DNA plasmids that were exposed to a pulsed electromagnetic field for 10-15 minutes in accordance with the present invention. It is to be noted that no transfection reagents were present with the DNA plasmids during exposure to the pulsed electromagnetic field.
4. The success of the transfection process was measured. The size of the DNA plasmids is measured using a Zetasizer Ultra (Malvern Panalytical, United Kingdom).

FIG. 3 illustrates the result data obtained for the measurement of the size of DNA plasmids with and without (control) being exposed to a pulsed electromagnetic field according to Experiment 4. It can be seen that the particle diameter of DNA plasmids which were not exposed to a pulsed electromagnetic field was measured at 15.03 nm, whereas the particle diameter of DNA plasmids which were exposed to a pulsed electromagnetic field was measured at 12.19 nm.

Thus, the size of the DNA plasmids can be reduced on exposure to the pulsed electromagnetic field compared to when no exposure to a pulsed electromagnetic field is used. This is likely to lead to higher frequencies of transfection.

### Experiment 5—Reduction of Particle Size in Cell Media

This experiment was undertaken to demonstrate a reduction in the size of particles in cell culture media on exposure of the media to a pulsed electromagnetic field according to the present invention.

The particle size of aggregates in the media was measured using a dynamic light scattering instrument, which in this example is a Zetasizer Ultra (Malvern Panalytical Ltd, UK), as per the protocol in Experiment 1.

The dynamic light scattering date relating to the control media (which was not exposed to a pulsed electromagnetic field) is shown in FIG. 4a. The dynamic light scattering data for the media that was exposed to a pulsed electromagnetic field in accordance with the present invention is shown in FIG. 4b. It can be seen the particle sizes in the media exposed to the pulsed electromagnetic field are reduced and becomes more uniform in their distribution compared to the particles of the control media. This is thought to be able to improve cells grown in the culture media which has been exposed to the pulsed electromagnetic field compared to controls where no pulsed electromagnetic field is used. It is also thought to be able to improve the transfection rates and efficiency and frequency thereof for which the cell media may be used.

Experiment 6—Influence of a Pulsed Electromagnetic Field on the Reconstitution of Lyophilised rhFVIII

A chromogenic assay was undertaken to measure the effect of applying a pulsed electromagnetic field using a device of the present invention to the activity levels of reconstituted lyophilized rhFVIII (Advate, Takeda, USA).

Chromogenic Factor VIII assays allow for the quantitative determination of FVIII in a sample. Factor X is first converted to Factor Xa (the rate of activation of Factor X is linearly related to the amount of FVIII). The quantification of Factor Xa activity is then measured with a synthetic chromogenic substrate. Factor Xa hydrolyses the chromogenic substrate releasing paranitroaniline (pNA) which is monitored kinetically at 405 nm and is proportional to the FVIII in the sample [3].

Three separate control experiments were carried out. The control experiments were not exposed to a pulsed electromagnetic field. These control experiments included the method steps of:
a) a Vial of 1000IU Advate (Takeda, USA) was reconstituted with 5 ml water for injection and left on a workbench at room temperature and pressure for 15 minutes.
b) An intermediate stock was made up by adding 10microlitres of the reconstituted Advate to 90 microlitres of FVIII deficient plasma sample (HemosIL FVIII deficient plasma-Instrumentation Laboratory, USA).
c) 50 microlitres of the intermediate stock was then taken and added to 950microlitres of the FVIII deficient plasma sample.

The activity value of the final neat solution made (to measure how pure the sample is) was expected to be 100% in the control experiments. However, the values given after running the chromogenic assay test three times on three different Advate vials was 77.9%, 86.4% and 89.4% for the control experiment.

Three separate “active” experiments using the pulsed electromagnetic field according to the present invention were carried out. The active experiments included the method steps of:
a) a Vial of 1000IU Advate (Takeda, USA) 18 was reconstituted with 5 ml water for injection and, with reference to FIG. 5, placed on a PulzFector (device which generates the pulsed electromagnetic field, St. Andrews Pharmaceuticals Technology Ltd, UK) 20 and surrounded by four Pulzar Pi devices 22 at room temperature and pressure for 15 minutes. The Pi devices 22 were arranged at equal distances apart from each other and the vial of Advate 18.
b) An intermediate stock was made up by adding 10microlitres of the reconstituted Advate to 90 microlitres of FVIII deficient plasma sample (HemosIL FVIII deficient plasma-Instrumentation Laboratory, USA).
c) 50 microlitres of the intermediate stock was then taken and added to 950microlitres of FVIII deficient plasma sample.

The activity value of the final neat solution was again expected to be 100% in the active experiments. The values given after running the chromogenic assay test three times on three different Advate vials was 98.7%, 97.1% and 108.1%.

After carrying out a t-Test, it was found that by applying a pulsed electromagnetic field to the active samples as per the present invention for the reconstitution process, there was a significant increase in the activity of a vial of 1000IU Advate.

TABLE 1
Neat Chromogenic Assay Results

| Control (No Pulsed | Active (Pulsed |
|---|---|
| Electromagnetic | Electromagnetic |
| Field Applied) (%) | Field Applied) (%) |
| 77.9 | 98.7 |
| 86.4 | 97.1 |
| 89.4 | 108.1 |

A=0.05
   t-Test: Paired Two Sample for Means

| | Control | Active |
|---|---|---|
| Mean | 84.5666667 | 101.3 |
| Variance | 35.583333 | 35.32 |
| Observations | 3 | 3 |
| Pearson Correlation | 0.59941178 | |
| Hypothesized Mean Difference | 0 | |
| df | 2 | |
| T Stat | −5.4382408 | |
| P (T <= t) one tail | 0.01609462 | |
| t Critical one tail | 2.91998558 | |
| P (T <= t) two tail | 0.03218924 | |
| t Critical two tail | 4.30265273 | |

### Experiment 7—Reconstitution of Freeze Dried Hemoleven (Factor XI)

Experiment 1 was repeated but with Hemoleven (Factor XI) in place of the Factor VIII.

The experiment was undertaken to measure the effect of use of the apparatus of the present invention on particle size of reconstituted freeze dried Factor XI to check the surprising observations on particle size were noted for agents other than just Factor VIII.

The particle size of the Factor XI exposed to a pulsed electromagnetic field is shown by the blue line (24) in FIGS. 6a and 6b in contrast to the particle size of the Factor XI control which is not exposed to a pulsed electromagnetic field (orange line (26)). It can be seen that there is a significant reduction in % volume of larger particles in the sample exposed to a pulsed electromagnetic field compared to the control. This mirrors the results seen with experiment 1. This shows the advantages of the present invention are not just limited to Factor VIII but are seen for other blood clotting factors as well.

FIG. 7 shows the data from experiment 7 with the particle size on the X axis and the number of particles per ml on the Y axis. The blue dots with the cross show the particle sizes for the Factor XI exposed to the pulsed electromagnetic field and the orange dots without a cross show the particle size for the Factor XI control. This shows the advantages of the present invention are not just limited to Factor VIII but are seen for other blood clotting factors as well.

### REFERENCES

[1]—“Native-like aggregates of Factor VIII (FVIII) are immunogenic von Willebrand Factor deficient and hemophilia A mice”—J Pharma Sci. 2012 June: 101 (6): 2055-2065
[2]—“Molecular aggravation of marketed recombinant FVIII products: Biochemical Evidence and Functional Effects”—TH Open 2019 April; 3 (2): e123-e131
[3]—“Diagnostics Directorate, North Glasgow Sector, Department of Hematology, Chromogenic FVIII”—LAP-GRI-COA-076—Revision No. 1, page 3-11 (NHSGGC)

1. A method of improving hydration and/or reducing the particle size of a product or agent, said method including the step of applying a pulsed electromagnetic field to the product or agent for a period of time sufficient to allow an increase in the hydration of the product or agent and/or a reduction of the particle size of the product or agent.

2. The method of claim 1, wherein the period of time to which the product or agent is exposed to the pulsed electromagnetic field is at least one of a pre-determined period of time, or a pre-determined period of time is 10-15 minutes+/−5 minutes.

3. (canceled)

4. The method according to claim 1, wherein the product or agent is any or any combination selected from the group consisting of media; cell media; a lyophilized or freeze-dried product or agent; a powdered or granular product or agent; a protein product such as a monoclonal antibody, a hormone, a fusion protein; a protein construct, trastuzumab; pembrolizumab; Infliximab; Daxibotulinumtoxin; Immunoglobulin; Omalizumab; Abatacept; Secukinumab; Interferon beta 1a; Bortezomib; DNA; DNA plasmid, Factor VIII; forms part of an intravenous formulation, product or agent; one that is known to be associated with CARPA reactions; is a dispersing agent, Tween 80, polysorbate 80.

5. The method according to claim 1, wherein the product or agent is a lyophilized or freeze-dried product or agent, the method includes the further step of reconstituting the lyophilized or freeze-dried product or agent by adding water or a liquid to the same to form a mixture; and applying the pulsed electromagnetic field to the mixture for the period of time or for the duration of reconstitution of the product or agent in the mixture.

6. (canceled)

7. The method according to claim 5, wherein the addition of the water or liquid to the product or agent to form the mixture takes place while at least one of the pulsed electromagnetic field is being applied to the water, liquid and/or mixture or the pulsed electromagnetic field is applied to the water or the liquid used for reconstitution prior to the addition with the lyophilized or freeze-dried product or agent.

8. (canceled)

9. The method according to claim 1, wherein the method includes selecting a step from the group consisting of: agitating the product, agent or mixture at any time prior to applying the pulsed electromagnetic field, during the step of applying the pulsed electromagnetic field and/or after the step of applying the pulsed electromagnetic field.

10. (canceled)

11. (canceled)

12. The method according to claim 1, wherein the pulsed electromagnetic field is applied to the product, agent or mixture during an infusion or injection process into a patient and/or during or before location in an intravenous bag or intravenous delivery vessel.

13. The method according to claim 1, wherein the pulsed electromagnetic field is generated by a member of the group consisting of: one or more electronic devices, electronic apparatus and/or circuits, transmission means or device, and one or more electronic transmission chips.

14. (canceled)

15. The method according to claim 13, wherein a plurality of transmission means or electronic transmission chips are provided which are arranged a pre-determined spaced distance apart from each other, and/or in a pre-determined pattern or array in order to provide the pulsed electromagnetic field, and/or there is one transmission means or electronic transmission chip per 105 to 115 cm² of a surface of the electronic device or circuit; or is 50 to 60 cm²; or is 25 to 30 cm²; or is 15 to 20 cm².

16. (canceled)

17. The method according to claim 1, wherein the pulsed electromagnetic field is provided at any or any combination of a frequency of 2.2-2.6 GHz; a frequency within the industrial, scientific and medical frequency band of 2.4 to 2.4835 GHz; wherein the field is pulsed at a frequency of approximately 50 MHz or less, 25 Hz or less, or 15 Hz or less; wherein each pulse of the pulsed electromagnetic field lasts for between approximately 1-20 ms or approximately 1 ms; wherein the time period between pulses is approximately 66 ms or less; or wherein the duty cycle of the pulsed electromagnetic field is less than 2%.

18. (canceled)

19. (canceled)

20. (canceled)

21. (canceled)

22. (canceled)

23. The method according to claim 1, wherein the transmission power of the transmission means or chip is 2 dBm-4 dBm, approximately 1 mW, approximately 2 mW or approximately 2.5119 mW.

24. Apparatus for improving hydration and/or reducing the particle size of a product or agent in use, said apparatus arranged to generate and emit a pulsed electromagnetic field capable of being directed towards a product or agent for a time sufficient to allow an increase in the hydration of the product or agent and/or a reduction of the particle size of the product or agent.

25. The apparatus of claim 24, wherein said apparatus includes any or any combination of agitation means for agitating the product, agent or a mixture in use; turbidity and/or particle size measuring means or device for measuring the turbidity and/or particle size of the product, agent or a mixture in use; audio, visual and/or kinaesthetic means for signalling to a user when the product, agent or a mixture being exposed to the pulsed electromagnetic field has a reached a certain level of hydration, turbidity and/or particle size.

26. The apparatus of claim 24, wherein the pulsed electromagnetic field is provided at a frequency of 2.2-2.6 GHz and/or the pulsed electromagnetic field is pulsed at a frequency of approximately 50 MHz or less, 25 Hz or less, or 15 Hz or less.

27. (canceled)

28. A reconstituted product or agent which has been reconstituted using the method of claim 1.

29. (canceled)

30. (canceled)
